SEQUENCE LISTING (35 sequences)

SEQ ID NO: 1 - protein, 9 aa, Artificial
  Synthetic peptide
DSSDSSDSS

SEQ ID NO: 2 - protein, 12 aa, Artificial
  Synthetic peptide
DSSDSSDSSDSS

SEQ ID NO: 3 - protein, 15 aa, Artificial
  Synthetic peptide
DSSDSSDSSDSSDSS

SEQ ID NO: 4 - protein, 18 aa, Artificial
  Synthetic peptide
DSSDSSDSSDSSDSSDSS

SEQ ID NO: 5 - protein, 21 aa, Artificial
  Synthetic peptide
DSSDSSDSSDSSDSSDSSDSS

SEQ ID NO: 6 - protein, 24 aa, Artificial
  Synthetic peptide
DSSDSSDSSDSSDSSDSSDSSDSS

SEQ ID NO: 7 - protein, 27 aa, Artificial
  Synthetic peptide
DSSDSSDSSDSSDSSDSSDSSDSSDSS

SEQ ID NO: 8 - protein, 30 aa, Artificial
  Synthetic peptide
DSSDSSDSSDSSDSSDSSDSSDSSDSSDSS

SEQ ID NO: 9 - protein, 33 aa, Artificial
  Synthetic peptide
DSSDSSDSSDSSDSSDSSDSSDSSDSSDSSDSS

SEQ ID NO: 10 - protein, 36 aa, Artificial
  Synthetic peptide
DSSDSSDSSDSSDSSDSSDSSDSSDSSDSSDSSDSS

SEQ ID NO: 11 - protein, 39 aa, Artificial
  Synthetic peptide
DSSDSSDSSDSSDSSDSSDSSDSSDSSDSSDSSDSSDSS

SEQ ID NO: 12 - protein, 42 aa, Artificial
  Synthetic peptide
DSSDSSDSSDSSDSSDSSDSSDSSDSSDSSDSSDSSDSSDSS

SEQ ID NO: 13 - protein, 45 aa, Artificial
  Synthetic peptide
DSSDSSDSSDSSDSSDSSDSSDSSDSSDSSDSSDSSDSSDSSDSS

SEQ ID NO: 14 - protein, 48 aa, Artificial
  Synthetic peptide
DSSDSSDSSDSSDSSDSSDSSDSSDSSDSSDSSDSSDSSDSSDSSDSS

SEQ ID NO: 15 - protein, 51 aa, Artificial
  Synthetic peptide
DSSDSSDSSDSSDSSDSSDSSDSSDSSDSSDSSDSSDSSDSSDSSDSSDSS

SEQ ID NO: 16 - protein, 54 aa, Artificial
  Synthetic peptide
DSSDSSDSSDSSDSSDSSDSSDSSDSSDSSDSSDSSDSSDSSDSSDSSDSSDSS

SEQ ID NO: 17 - protein, 57 aa, Artificial
  Synthetic peptide
DSSDSSDSSDSSDSSDSSDSSDSSDSSDSSDSSDSSDSSDSSDSSDSSDSSDSSDSS

SEQ ID NO: 18 - protein, 60 aa, Artificial
  Synthetic peptide
DSSDSSDSSDSSDSSDSSDSSDSSDSSDSSDSSDSSDSSDSSDSSDSSDSSDSSDSSDSS

SEQ ID NO: 19 - protein, 12 aa, Artificial
  Synthetic peptide
DSSNSSDSSNSS

SEQ ID NO: 20 - protein, 5 aa, Artificial
  Synthetic peptide
PSGSP

SEQ ID NO: 21 - protein, 5 aa, Artificial
  Synthetic peptide
ASASA

SEQ ID NO: 22 - protein, 15 aa, Artificial
  Synthetic peptide
GGGGSGGGGSGGGGS

SEQ ID NO: 23 - protein, 4 aa, Artificial
  Synthetic peptide
GGGG

SEQ ID NO: 24 - protein, 6 aa, Artificial
  Synthetic peptide
GGSGGS

SEQ ID NO: 25 - protein, 5 aa, Artificial
  Synthetic peptide
PSPSP

SEQ ID NO: 26 - protein, 5 aa, Artificial
  Synthetic peptide
ASASA

SEQ ID NO: 27 - protein, 5 aa, Artificial
  Synthetic peptide
PSPSP

SEQ ID NO: 28 - protein, 4 aa, Artificial
  Synthetic peptide
KKKK

SEQ ID NO: 29 - protein, 4 aa, Artificial
  Synthetic peptide
RRRR

SEQ ID NO: 30 - protein, 5 aa, Artificial
  Synthetic peptide
GGGGS

SEQ ID NO: 31 - protein, 10 aa, Artificial
  Synthetic peptide
GGGGSGGGGS

SEQ ID NO: 32 - protein, 15 aa, Artificial
  Synthetic peptide
GGGGSGGGGSGGGGS

SEQ ID NO: 33 - protein, 20 aa, Artificial
  Synthetic peptide
GGGGSGGGGSGGGGSGGGGS

SEQ ID NO: 34 - protein, 25 aa, Artificial
  Synthetic peptide
GGGGSGGGGSGGGGSGGGGSGGGGS

SEQ ID NO: 35 - protein, 30 aa, Artificial
  Synthetic peptide
GGGGSGGGGSGGGGSGGGGSGGGGSGGGGS

(19) United States
(12) Patent Application Publication
(10) Pub. No.: US 2014/0314691 A1
(43) Pub. Date: Oct. 23, 2014

(54) COMPOSITIONS FOR IMPROVING THE APPEARANCE AND/OR HEALTH OF TEETH

(71) Applicant: C3 JIAN, INC., MARINA DEL REY, CA (US)

(72) Inventors: Randal H. ECKERT, Rancho Palos, CA (US); Daniel K. YARBROUGH, Ann Arbor, MI (US); Brian C. VARNUM, Santa Monica, CA (US)

(73) Assignee: C3 JIAN, INC., MARINA DEL REY, CA (US)

(21) Appl. No.: 14/205,098

(22) Filed: Mar. 11, 2014

Related U.S. Application Data
(60) Provisional application No. 61/785,166, filed on Mar. 14, 2013.
(60) Provisional application No. 61/883,016, filed on Sep. 26, 2013.

(51) Int. Cl. A61K 8/64; A61K 8/66; A61K 8/20; A61K 8/25; A61K 8/24; A61K 8/28; A61K 8/29; A61Q 11/00; A61K 8/22

(52) U.S. Cl. CPC A61K 8/64; A61Q 11/00; A61K 8/66; A61K 8/20; A61K 8/22; A61K 8/24; A61K 8/28; A61K 8/29; A61K 8/25
USPC 424/50; 424/49; 424/53; 424/57

(57) ABSTRACT

In various embodiments compositions are provided for the whitening, and/or brightening, and/or restoration of a tooth. In various embodiments the compositions, referred to herein as “dental care” compositions typically comprise a binding moiety that binds to calcium and/or to tooth enamel and/or to pellicle attached to an active agent. The active agent is selected to deliver a particular activity (e.g., whitening, remineralization, desensitization, brightening, etc.) to the tooth.

### CROSS-REFERENCE TO RELATED APPLICATIONS

This application claims benefit of and priority to U.S. Ser. No. 61/785,166, filed on Mar. 14, 2013, and to U.S. Ser. No. 61/883,016, filed on Sep. 26, 2013, both of which are incorporated herein by reference in their entirety for all purposes.

### STATEMENT OF GOVERNMENTAL SUPPORT

[Not Applicable]

### BACKGROUND

The tooth is a structure having layers of soft inner media covered by a hard outer surface. A pulp chamber maintains the blood and nerve supply and is protected by the hard dentin layer, which attenuates shock and pressure experienced during mastication or in the event of a traumatic event. An outer layer of enamel covers the dentin. The inorganic mineral content in dentin is quite low compared to enamel, with the calcium weight fractions in dentin about 28% lower than in enamel. The decreased mineral content in dentin is compensated for by an equivalently elevated weight fraction of organic constituents. Although the Ca:P ratios in both calcium and dentin are about the same, the specific surface areas differ markedly.

Tooth enamel is comprised of a heterogeneous arrangement of inorganic mineral and less than 1% organic substances, including proteins and collagen. The overall thickness of human enamel can be up to several millimeters and visually, the apatite crystals bear a hexagonal arrangement, although the actual unit cell pertaining to hydroxyapatite is likely comprised of two different sets of monoclinic crystal arrangements, with one monoclinic arrangement arising from two hexagonal cells. The apatite crystals found in enamel predominantly manifest hydroxyl or carbonate species. Additionally, these crystals may also have magnesium, strontium, and sodium substituted for calcium.

Tooth enamel (and other tooth components) typically show deleterious changes over time. Such changes include, but are not limited to dissolution of the enamel, formation of dental caries, increase of dental sensitivity, discoloration of the tooth surface, and the like. While numerous means are known to address these changes, they are typically of limited efficacy and short duration.

### SUMMARY

In various embodiments compositions are provided for the whitening, and/or brightening, and/or restoration of a tooth. In various embodiments the compositions, referred to herein as “dental care” compositions typically comprise a binding moiety that binds to calcium and/or to tooth enamel and/or to pellicle attached to an active agent. The active agent is selected to deliver a particular activity (e.g., whitening, remineralization, desensitization, brightening, etc.) to the tooth.

In various aspects, the invention(s) contemplated herein may include, but need not be limited to, any one or more of the following embodiments:

Embodiment 1: A composition for the whitening, and/or brightening, and/or restoration of a tooth, said composition including: a binding moiety that binds to calcium and/or to tooth enamel and/or to pellicle attached to an active agent selected from the group consisting of a colorant, a whitening agent, an enzyme, an anti-plaque agent, a desensitization agent, a remineralization agent, or any combination thereof.

Embodiment 2: The composition of embodiment 1, wherein said active agent is selected from the group consisting of a white colorant, a whitening agent other than a fluorescent molecule, an enzyme, an anti-plaque agent, a desensitization agent, a remineralization agent, or any combination thereof.

Embodiment 3: The composition according to any one of embodiments 1-2, wherein a plurality of binding moieties are attached to each active agent.

Embodiment 4: The composition according to any one of embodiments 1-2, wherein a plurality of active agents are attached to each binding moiety.

Embodiment 5: The composition according to any one of embodiments 1-2, wherein a single binding moiety is attached to a single active agent.

Embodiment 6: The composition according to any one of embodiments 1-5, wherein said binding moiety is selected from the group consisting of an enamel matrix proteins (EMP) or enamel-binding fragment thereof, an enamel or calcium binding peptide, and an enamel or calcium binding tannin.

Embodiment 7: The composition of embodiment 6, wherein said binding moiety includes an enamel matrix protein or a calcium or enamel binding fragment thereof.

Embodiment 8: The composition of embodiment 7, wherein said binding moiety compromises amelogenin or ameloblastin or an enamel or calcium binding fragment thereof.

Embodiment 9: The composition of embodiment 6, wherein said binding moiety includes an enamel and/or calcium binding peptide including one or more peptide domains said domain(s) including the sequence (X-Y-Z)ₙ, wherein: X is an amino acid selected from the group consisting of aspartic acid, glutamic acid, asparagine, alanine and glutamine or a conservative substitution thereof; Y and Z are amino acids independently selected from alanine, serine, threonine, phosphoserine, and phosphothreonine, or conservative substitutions thereof; and n is a number from 1 to 100; and wherein when more than one of said domains is present the domains can be the same or different; and wherein said calcium-binding peptide moiety binds calcium phosphate.

Embodiment 10: The composition of embodiment 9, wherein said calcium-binding peptide includes two or more of said domains joined by a non-peptide linker.

Embodiment 11: The composition of embodiment 9, wherein said calcium-binding peptide includes two or more of said domains joined by a peptide linker.

Embodiment 12: The composition of embodiment 9, wherein said peptide includes one copy of the sequence (X-Y-Z)ₙ.

Embodiment 13: The composition of embodiment 12, wherein said peptide consists of the sequence (X-Y-Z)ₙ.

Embodiment 14: The composition according to any one of embodiments 9-13, wherein Y and Z are the same amino acid.

Embodiment 15: The composition of embodiment 14, wherein Y and Z are both serine or a conservative substitution thereof.

Embodiment 16: The composition of embodiment 15, wherein Y and Z are both serine.

Embodiment 17: The composition of embodiment 14, wherein Y and Z are both phosphoserine or a conservative substitution thereof.

Embodiment 18: The composition of embodiment 17, wherein Y and Z are both phosphoserine.

Embodiment 19: The composition of embodiment 14, wherein Y and Z are both threonine or a conservative substitution thereof.

Embodiment 20: The composition of embodiment 19, wherein Y and Z are both threonine.

Embodiment 21: The composition of embodiment 14, wherein Y and Z are both phosphothreonine or a conservative substitution thereof.

Embodiment 22: The composition of embodiment 21, wherein Y and Z are both phosphothreonine.

Embodiment 23: The composition of embodiment 14, wherein Y and Z are both alanine or a conservative substitution thereof.

Embodiment 24: The composition of embodiment 23, wherein Y and Z are both alanine.

Embodiment 25: The composition according to any one of embodiments 9-24, wherein X is aspartic acid or a conservative substitution thereof.

Embodiment 26: The composition of embodiment 25, wherein X is aspartic acid.

Embodiment 27: The composition according to any one of embodiments 9-24, wherein X is glutamic acid or a conservative substitution thereof.

Embodiment 28: The composition of embodiment 27, wherein X is glutamic acid.

Embodiment 29: The composition according to any one of embodiments 9-24, wherein X is asparagine or a conservative substitution thereof.

Embodiment 30: The composition of embodiment 29, wherein X is asparagine.

Embodiment 31: The composition according to any one of embodiments 9-24, wherein X is glutamine or a conservative substitution thereof.

Embodiment 32: The composition of embodiment 31, wherein X is glutamine.

Embodiment 33: The composition according to any one of embodiments 9-24, wherein X is alanine or a conservative substitution thereof.

Embodiment 34: The composition of embodiment 33, wherein X is alanine.

Embodiment 35: The composition according to any one of embodiments 9-34, wherein said peptide ranges in length from about 6 to about 60 amino acids.

Embodiment 36: The composition of embodiment 35, wherein said peptide ranges in length from about 6 to about 36 amino acids.

Embodiment 37: The composition according to any one of embodiments 9-36, wherein n ranges from about 3 up to about 12.

Embodiment 38: The composition according to any one of embodiments 9-36, wherein n ranges from about 3 up to about 8.

Embodiment 39: The composition of embodiment 9, wherein the amino acid sequence of said peptide includes a sequence selected from the group consisting (DSS)₃ (SEQ ID NO:1), (DSS)₄ (SEQ ID NO:2), (DSS)₅ (SEQ ID NO:3), (DSS)₆ (SEQ ID NO:4), (DSS) (SEQ ID NO:5), (DSS)₈ (SEQ ID NO:6), (DSS)₉ (SEQ ID NO:7), (DSS)₁₀ (SEQ ID NO:8), (DSS)₁₁ (SEQ ID NO:9), (DSS)₁₂ (SEQ ID NO:10), (DSS)₁₃ (SEQ ID NO:11), (DSS)₁₄ (SEQ ID NO:12), (DSS)₁₅ (SEQ ID NO:13), (DSS)₁₆ (SEQ ID NO:14), (DSS)₁₇ (SEQ ID NO:15), (DSS)₁₈ (SEQ ID NO:16), (DSS)₁₉ (SEQ ID NO:17), (DSS)₂₀ (SEQ ID NO:18).

Embodiment 40: The composition of embodiment 39, wherein said peptide ranges in length up to about 60 amino acids.

Embodiment 41: The composition of embodiment 39, wherein said peptide ranges in length up to about 30 amino acids.

Embodiment 42: The composition of embodiment 9, wherein the amino acid sequence of said peptide consists of a sequence selected from the group consisting of (DSS)₃ (SEQ ID NO:1), (DSS)₄ (SEQ ID NO:2), (DSS)₅ (SEQ ID NO:3), (DSS)₆ (SEQ ID NO:4), (DSS)₇ (SEQ ID NO:5), (DSS)₈ (SEQ ID NO:6), (DSS)₉ (SEQ ID NO:7), (DSS)₁₀ (SEQ ID NO:8), (DSS)₁₁ (SEQ ID NO:9), (DSS)₁₂ (SEQ ID NO:10), (DSS)₁₃ (SEQ ID NO:11), (DSS)₁₄ (SEQ ID NO:12), (DSS)₁₅ (SEQ ID NO:13), (DSS)₁₆ (SEQ ID NO:14), (DSS)₁₇ (SEQ ID NO:15), (DSS)₁₈ (SEQ ID NO:16), (DSS)₁₉ (SEQ ID NO:17), (DSS)₂₀ (SEQ ID NO:18).

Embodiment 43: The composition according to any one of embodiments 9-16, 19, 20, and 23-42 wherein said peptide is not phosphorylated.

Embodiment 44: The composition according to any one of embodiments 9-42, wherein said peptide is partially phosphorylated.

Embodiment 45: The composition according to any one of embodiments 9-42, wherein said peptide is fully phosphorylated.

Embodiment 46: The composition according to any one of embodiments 9-45, wherein said peptide is an L peptide.

Embodiment 47: The composition according to any one of embodiments 9-45, wherein said peptide is a D peptide.

Embodiment 48: The composition according to any one of embodiments 9-45, wherein said peptide is a beta peptide.

Embodiment 49: The composition according to any one of embodiments 1-48, wherein said active agent comprises a whitening agent and/or a colorant.

Embodiment 50: The composition of embodiment 49, wherein said whitening agent and/or colorant includes a bleach.

Embodiment 51: The composition of embodiment 50, wherein said whitening agent and/or colorant includes a peroxide.

Embodiment 52: The composition of embodiment 49, wherein said whitening agent and/or colorant is selected from the group consisting of hydroxyapatite, zirconium silicate, titanium dioxide, titanium dioxide nanoparticles silica dioxide, a fluorescent molecule, and a pigment or dye.

Embodiment 53: The composition of embodiment 49, wherein said whitening agent and/or colorant is selected from the group consisting of hydroxyapatite, zirconium silicate, titanium dioxide, titanium dioxide nanoparticles silica dioxide, and a pigment or dye.

Embodiment 54: The composition of embodiment 49, wherein said whitening agent and/or colorant is a pigment or dye selected from the group consisting of FITC, Rhodamine, Blue No. 1, Yellow No. 5.

Embodiment 55: The composition of embodiment 49, wherein said whitening agent and/or colorant is a fluorescent or luminescent molecule selected from the group consisting of luminol, and isoluminol.

Embodiment 56: The composition according to any one of embodiments 1-55, wherein said active agent includes an enzyme.

Embodiment 57: The composition of embodiment 56, wherein said active agent includes an enzyme is selected from the group consisting of an oxidase, a peroxidase, a protease, a lipase, a glycosidase, an esterase, and a polysaccharide hydrolase.

Embodiment 58: The composition according to any one of embodiments 1-57, wherein said active agent includes an anti-plaque agent.

Embodiment 59: The composition of embodiment 58, wherein said antiplaque agents is selected from the group consisting of fluoride ion sources and non-peptide antimicrobial agents.

Embodiment 60: The composition according to any one of embodiments 1-59, wherein said active agent includes a desensitization agent.

Embodiment 61: The composition of embodiment 60, wherein said desensitization includes an agent selected from the group consisting of dipotassium oxalate, 2-hydroxyethyl methacrylate, and potassium nitrate.

Embodiment 62: The composition according to any one of embodiments 1-61, wherein said active agent includes a remineralization agent.

Embodiment 63: The composition of embodiment 62, wherein said remineralization agent includes an agent selected from the group consisting of a water-soluble phosphate and/or fluoride salts, a water-soluble calcium salt, a water-soluble salt of a divalent metal other than calcium, an antioxidant (e.g. coenzyme Q10), or any combination thereof.

Embodiment 64: The composition according to any one of embodiments 1-63, wherein said active agent is attached to said binding moiety through a covalent linkage.

Embodiment 65: The composition according to any one of embodiments 1-63, wherein said active agent is attached to said binding moiety through a non-covalent linkage.

Embodiment 66: The composition of embodiment 65, wherein said non-covalent linkage includes biotin and avidin, biotin and streptavidin, a streptavidin tag and streptavidin, maltose binding protein and maltose, maltose binding protein and amylase, a polyhistidine tag and an polyHis affinity ion, glutathione S-transferase and glutathione, an epitope tag and an antibody, and combinations thereof.

Embodiment 67: The composition according to any one of embodiments 1-66, wherein said active agent is attached directly to said binding moiety.

Embodiment 68: The composition according to any one of embodiments 1-66, wherein said active agent is attached to said binding moiety through a linker.

Embodiment 69: The composition of embodiment 68, wherein said active agent is attached to said binding moiety through a peptide linker.

Embodiment 70: The composition of embodiment 69, wherein said peptide linker ranges in length from about 2 to about 25 amino acids, or ranges in length from about 3 to about 20 amino acids, or ranges in length from about 3 to about 15 amino acids, or ranges in length up to about 10 amino acids.

Embodiment 71: The composition according to any one of embodiments includes 69-70, wherein the linker is a peptide including amino acids selected from the group consisting of glycine, alanine, serine, and mixtures thereof.

Embodiment 72: The composition according to any one of embodiments includes 69-70, wherein said peptide linker includes or consists of the amino acid sequence of a peptide linker shown in Table 2.

Embodiment 73: The composition of embodiment 68, wherein said active agent is attached to said binding moiety through a non-peptide linker.

Embodiment 74: The composition of embodiment 73, wherein said non-peptide linker includes is selected from the group consisting of ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, and cetyl alkyl chains.

Embodiment 75: The composition of embodiment 73, wherein said non-peptide linker includes a non-peptide linker shown in Table 2.

Embodiment 76: The composition according to any one of embodiments 1-75, wherein the binding moiety expressly excludes peptides described in U.S. Pat. No. 7,807,141 B2 and U.S. Pat. No. 8,354,381.

Embodiment 77: The composition according to any one of embodiments 1-76, wherein the active agent expressly excludes fluorescent or luminescent materials.

Embodiment 78: The composition according to any one of embodiments 1-76, wherein the active agent expressly excludes fluorescein or carboxyfluorescein.

Embodiment 79: The composition according to any one of embodiments 1-78, wherein the active agent expressly excludes a dye or colorant other than a white dye or colorant 80: The composition according to any one of embodiments 1-78, wherein the active agent expressly excludes a dye or colorant.

Embodiment 81: An oral care formulation including an effective amount of the composition according to any one of embodiments 1-80.

Embodiment 82: The oral care formulation of embodiment 81, wherein said formulation is selected from the group consisting of toothpaste, dental cream, gel, tooth powder, mouth wash, breath freshener, and dental floss.

Embodiment 83: The oral care formulation according to any one of embodiments 81-82, where said formulation includes a reagent selected from the group consisting of abrasives, surfactants, chelating agents, fluoride sources, thickening agents, buffering agents, solvents, humectants, carriers, and bulking agents.

Embodiment 84: The oral care formulation according to any one of embodiments 81-83, wherein said formulation includes a flavorant.

Embodiment 85: The oral care formulation according to any one of embodiments 81-84, wherein said formulation includes a flavorant selected from the group consisting of oil of wintergreen, oil of peppermint, oil of spearmint, menthol, methyl salicylate, eucalyptol, and vanillin.

Embodiment 86: A method of improving the health and/or appearance of a tooth, said method including contacting said tooth with an effective amount of a composition according to any one of embodiments 1-80 and/or a formulation according to any one of embodiments 81-85.

Embodiment 87: The method of embodiment 86, wherein said improving the health and/or appearance of a tooth includes brightening and/or whitening said tooth and the active agent including the composition and/or formulation includes a whitening and/or brightening agent.

Embodiment 88: The method according to any one of embodiments 86-87, wherein said improving the health and/or appearance of a tooth includes remineralizing and/or preventing demineralization of a tooth and the active agent including the composition and/or formulation includes a remineralizing agent.

Embodiment 89: The method according to any one of embodiments 86-87, wherein said improving the health and/or appearance of a tooth includes desensitizing a tooth and the active agent including the composition and/or formulation includes a desensitizing agent.

Embodiment 90: The method according to any one of embodiments 86-89, wherein said improving the health and/or appearance of a tooth includes reducing plaque formation and/or removing plaque and the active agent including the composition and/or formulation includes an anti-plaque agent.

Embodiment 91: The method according to any one of embodiments 86-90, wherein said tooth is a tooth in a human.

Embodiment 92: The method of embodiment 91, wherein said method is performed by said human.

Embodiment 93: The method of embodiment 91, wherein said method is performed by a dentist to a dental patient.

In certain embodiments the binding moiety expressly excludes peptides described in U.S. Pat. No. 7,807,141 B2 and U.S. Pat. No. 8,354,381 which are incorporated herein by reference for the peptides disclosed therein. In certain embodiments, the active agent(s) expressly exclude fluorescent or luminescent materials. In certain embodiments the active agent(s) expressly exclude fluoresceine or carboxyfluorescein. in certain embodiments the active agent(s) expressly exclude a dye or colorant other than a white dye or colorant.

### DEFINITIONS

The term “calcium-binding moiety” includes calcium-binding peptides as well as non-peptide calcium-binding molecules as described herein.

The term “peptide” as used herein refers to a polymer of amino acid residues typically ranging in length from 2 to about 50, 80, or about 100 residues. In certain embodiments the peptide ranges in length from about 2, 3, 4, 5, 7, 9, 10, or 11 residues to about 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, phosphonamide, carbomate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542; and Kaltenbronn et al., (1990) Pp. 969-970 in Proc. 11th American Peptide Symposium, ESCOM Science Publishers, The Netherlands, and the like)).

The term “residue”” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

“DSS peptides”, as used herein, include calcium-binding peptides comprising repeats of the Asp-Ser-Ser motif (e.g., as described in PCT Publication WO 2007/038683) and/or variations thereof, e.g., as described herein. In certain embodiments “DSS peptides” include calcium-binding peptides/proteins having multiple domains comprising repeats of the Asp-Ser-Ser motif and variations thereof “DSS” peptides can also comprise conjugates wherein two or more domains comprising repeats of the Asp-Ser-Ser motif and variations thereof are attached by non-peptide linkers thereby forming a multi-domain conjugate.

“β-peptides” comprise of “β amino acids”, which have their amino group bonded to the β carbon rather than the α-carbon as in the 20 standard biological amino acids. The only commonly naturally occurring β amino acid is β-alanine.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids).

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. A compound of the invention “corresponds” to a natural peptide if it elicits a biological activity (e.g., Ca binding activity) similar to that of the naturally occurring peptide. The elicited activity may be the same as, greater than or less than that of the natural peptide. In general, a peptoid will have an essentially corresponding monomer sequence, where a natural amino acid is replaced by an N-substituted glycine derivative, if the N-substituted glycine derivative resembles the original amino acid in hydrophilicity, hydrophobicity, polarity, etc. The following are illustrative, but non-limiting N-substituted glycine replacements: N-(1-methylprop-1-yl)glycine substituted for isoleucine (Ile), N-(prop-2-yl)glycine for valine (Val), N-benzylglycine for phenylanlaine (Phe), N-(2-hydroxyethyl)glycine for serine (Ser), and the like. In certain embodiments substitutions need not be “exact”. Thus for example, in certain embodiments N-(2-hydroxyethyl)glycine may substitute for Ser, Thr, Cys, and/or Met; N-(2-methylprop-1-yl)glycine may substitute for Val, Leu, and/or Ile. In certain embodiments N-(2-hydroxyethyl)glycine can be used to substitute for Thr and Ser, despite the structural differences: the side chain in N-(2-hydroxyethyl)glycine is one methylene group longer than that of Ser, and differs from Thr in the site of hydroxy-substitution. In general, one may use an N-hydroxyalkyl-substituted glycine to substitute for any polar amino acid, an N-benzyl- or N-aralkyl-substituted glycine to replace any aromatic amino acid (e.g., Phe, Trp, etc.), an N-alkyl-substituted glycine such as N-butylglycine to replace any nonpolar amino acid (e.g., Leu, Val, Ile, etc.), and an N-(aminoalkyl)glycine derivative to replace any basic polar amino acid (e.g., Lys and Arg).

Where an amino acid sequence is provided herein, L-, D-, or beta amino acid versions of the sequence are also contemplated as well as retro, inversion, and retro-inversion isoforms. In addition, conservative substitutions are contemplated. Non-protein backbones, such as PEG, alkane, ethylene bridged, ester backbones, and other backbones are also contemplated. Also fragments ranging in length from about 5, 6, 7, 8, 9, 10, 11, 12, 13, 14, 15, 16, 17, 18, 19, 20, 21, 22, 23, 24, or 25 amino acids up to the full length minus one amino acid of the peptide are contemplated where the fragment retains at least 50%, preferably at least 60% 70% or 80%, more preferably at least 90%, 95%, 98%, 99%, or at least 100% of the activity (e.g., binding specificity and/or avidity) of the corresponding unsubstituted peptide are contemplated.

In certain embodiments, conservative substitutions of the amino acids comprising any of the sequences described herein are contemplated. In various embodiments one, two, three, four, or five different residues are substituted. The term “conservative substitution” is used to reflect amino acid substitutions that do not substantially alter the activity (e.g., antimicrobial activity and/or specificity) of the molecule. Typically conservative amino acid substitutions involve substitution one amino acid for another amino acid with similar chemical properties (e.g. charge or hydrophobicity). Certain conservative substitutions include “analog substitutions” where a standard amino acid is replaced by a non-standard (e.g., rare, synthetic, etc) amino acid differing minimally from the parental residue. Amino acid analogs are considered to be derived synthetically from the standard amino acids without sufficient change to the structure of the parent, are isomers, or are metabolite precursors. Examples of such “analog substitutions include but are not limited to 1) Lys-Orn, 2) Leu-Norleucine, 3) Lys-Lys[TFA], 4) Phe-Phe[Gly], and 5) δ-amino butylglycine-ξ-amino hexylglycine, where Phe[gly] refers to phenylglycine (a Phe derivative with a H rather than CH₃ component in the R group), and Lys[TFA] refers to a Lys where a negatively charged ion (e.g., TFA) is attached to the amine R group. Other conservative substitutions include “functional substitutions” where the general chemistries of the two residues are similar, and can be sufficient to mimic or partially recover the function of the native peptide. Strong functional substitutions include, but are not limited to 1) Gly/Ala, 2) Arg/Lys, 3) Ser/Tyr/Thr, 4) Leu/Ile/Val, 5) Asp/Glu, 6) Gln/Asn, and 7) Phe/Trp/Tyr, while other functional substitutions include, but are not limited to 8) Gly/Ala/Pro, 9) Tyr/His, 10) Arg/Lys/His, 11) Ser/Thr/Cys, 12) Leu/Ile/Val/Met, and 13) Met/Lys (special case under hydrophobic conditions). Various “broad conservative substations” include substitutions where amino acids replace other amino acids from the same biochemical or biophysical grouping. This is similarity at a basic level and stems from efforts to classify the original 20 natural amino acids. Such substitutions include 1) nonpolar side chains: Gly/Ala/Val/Leu/Ile/Met/Pro/Phe/Trp, and/or 2) uncharged polar side chains Ser/Thr/Asn/Gln/Tyr/Cys. In certain embodiments broad-level substitutions can also occur as paired substitutions. For example, Any hydrophilic neutral pair [Ser, Thr, Gln, Asn, Tyr, Cys]+[Ser, Thr, Gln, Asn, Tyr, Cys] can may be replaced by a charge-neutral charged pair [Arg, Lys, His]+[Asp, Glu]. The following six groups each contain amino acids that, in certain embodiments, are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V); and 6) Phenylalanine (F), Tyrosine (Y), Tryptophan (W). Where amino acid sequences are disclosed herein, amino acid sequences comprising, one or more of the above-identified conservative substitutions are also contemplated.

In certain embodiments, peptides compromising at least 80%, preferably at least 85% or 90%, and more preferably at least 95% or 98% sequence identity with any of the sequences described herein (and preferably retain at least 50%, 60%, 70%, 80%, 90% 95%, 98% or 100% or more binding specificity and/or avidity of the unmodified peptide) are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides of this invention sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman (1981) Adv. Appl. Math. 2: 482, by the homology alignment algorithm of Needleman & Wunsch (1970) J. Mol. Biol. 48: 443, by the search for similarity method of Pearson & Lipman (1988) Proc. Natl. Acad. Sci., USA, 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “consisting essentially of” when used with respect to an calcium-binding peptide or other moiety as described herein, indicates that the peptide or other moiety encompassed by variants, analogues, or derivatives thereof possess substantially the same or greater binding activity and/or specificity and/or avidity as the referenced moiety.

The terms “isolated” “purified” or “biologically pure” refer to material which is substantially or essentially free from components that normally accompany it as found in its native state. In the case of a peptide, an isolated (naturally occurring) peptide is typically substantially free of components with which it is associated in the cell, tissue, or organism. The term isolated also indicates that the peptide is not present in a phage display, yeast display, or other peptide library.

The term “tooth surface” will refer to a surface comprised of tooth enamel (typically exposed after professional cleaning or polishing) or tooth pellicle (an acquired surface comprising salivary glycoproteins).

As used herein, the terms “pellicle” and “tooth pellicle” will refer to the thin film (typically ranging from about 1 μm to about 200 μm thick) derived from salivary glycoproteins which forms over the surface of the tooth crown. Daily tooth brushing tends to only remove a portion of the pellicle surface while abrasive tooth cleaning and/or polishing (typically by a dental professional) will expose more of the tooth enamel surface.

As used herein, the terms “enamel” and “tooth enamel” will refer to the highly mineralized tissue that forms the outer layer of the tooth. The enamel layer is composed primarily of crystalline calcium phosphate (i.e. hydroxyapatite) along with water and some organic material. In one embodiment, the tooth surface is selected from the group consisting of tooth enamel and tooth pellicle. As used herein, the term “tooth-binding peptide” will refer to a peptide that binds to tooth enamel or tooth pellicle.

The terms “coupling”, “coupled”, and attached as used herein refer to any chemical association and includes both covalent and noncovalent interactions.

### BRIEF DESCRIPTION OF THE DRAWINGS

[Not Applicable]

### DETAILED DESCRIPTION

In various embodiments compositions are provided for the whitening, and/or brightening, and/or restoration of a tooth. In various embodiments the compositions, referred to herein as “dental care” compositions typically comprise a binding moiety that binds to calcium and/or to tooth enamel and/or to pellicle attached to an active agent. The active agent is selected to deliver a particular activity (e.g., whitening, remineralization, desensitization, brightening, etc.) to the tooth.

Without being bound to a particular theory, it is believed that the binding moiety, by interacting with a component of (or on) the tooth (e.g., pellicle, enamel, calcium, etc.) can bind the active agent to the tooth and/or increase the interaction of the active agent with the tooth, and/or increase the concentration and/or duration of the association of the active agent with the tooth. As a consequence, the dental care compositions described herein are believed to provide greater efficacy (e.g., greater efficacy at a given dose and/or equal efficacy at a lower dose, and/or greater persistence of beneficial effect) than previous dental care compositions).

In various embodiments the dental care compositions can be applied directly to the tooth surface (e.g., via a swab, rinse, aerosol, tooth “whitening” strip, etc.) or the compositions can be formulated into various home care products (e.g., toothpaste, dental cream, gel, tooth powder, mouth wash, breath freshener, dental floss, etc.).

### Components of a Dental Care Composition.

In various embodiments, the dental care compositions described herein comprise a binding moiety that binds and/or interacts with a tooth or a component of a tooth attached to an active agent that, e.g., provides a desirable action on the tooth.

Binding Moiety.

In various embodiments the dental care compositions comprise a binding moiety that binds to a tooth or component thereof. In certain embodiments the binding moiety binds calcium, and/or hydroxyapatite, and/or calcium phosphate, and/or tooth enamel, and/or dental pellicle, and/or dentin.

Any of a variety of binding moieties are contemplated. In certain embodiments the binding moieties include, for example alendronate (see, e.g., Chen et al. (2009) Antimicrobial Agents Chemother., 53(11): 4898-4902), bisphosphonates (e.g., pamidronate, zoledronic acid and the like), inorganic calcium/enamel binding molecules, chlorhexidine, CPC, tetracycline, fluorides, enamel/calcium bining tannins, and enamel/calcium-binding peptides/proteins.

Illustrative enamel-binding peptides include, but are not limited to those produced or derived from the sequence of enamel matrix proteins (EMPs), like amelogenin or ameloblastin or enamel-binding fragments thereof. Also contemplated are dentinsialoprotein or enamel-binding fragments thereof.

In certain embodiments, the binding moieties comprise a calcium (or enamel binding) peptides. Calcium-binding peptides are moieties that comprise or consist of one or more calcium-binding peptide domains (e.g., DSS peptide domains such as those described in PCT Publication WO 2007/038683 which is incorporated herein by reference for the calcium-binding peptides described herein). Where multiple calcium-binding peptide domains are present they can be chemically conjugated together or can be directly attached via, for example a peptide bond or joined, for example via a peptide linker to form multi-domain calcium-binding peptides.

In certain embodiments, a series of calcium-binding peptides made up of variations of the Asp-Ser-Ser motif (“DSS” peptides) are provided as well as moieties comprising multiple calcium-binding peptides. These “DSS” peptides have been shown to bind tightly and specifically to calcium phosphate surfaces. In addition, these peptides have been shown to recruit calcium phosphate to such surfaces and can serve as binding moieties for the attachment of fluorescent labels to calcified surfaces regardless of their phosphorylation state.

In various embodiments the peptides/peptide domains described herein, referred to generally as DSS peptides, can be composed of various numbers and/or combinations of a 3 amino acid subunit: the three amino acid Asp-Ser-Ser motif of DPP or variations thereof. Examples of three amino acid repeats that may be utilized include, but are not limited to, Asp-Ser-Ser (DSS), Glu-Ser-Ser (ESS), Asp-Thr-Thr (DTT), Glu-Thr-Thr (ETT), Asn-Ser-Ser (NSS), Asn-Thr-Thr (NTT), Gln-Ser-Ser (QSS), Gln-Thr-Thr (QTT), and variations thereof. Alternatively or in addition to these repeat sequences, the peptides disclosed herein may include minor variations of these repeats, including but not limited to Asp-Ser-Thr (DST), Asp-Ala-Ala (DAA), or Ala-Ser-Thr (AST), and the like. In various embodiments one or more amino acid residues within a three amino acid repeat may be chemically modified. For example, in certain embodiments the peptides can contain one or more Ser or Thr residues in which a hydroxyl group has been modified by the addition of a phosphate group (pSer, pThr), etc. In various embodiments the peptides vary in length from three to greater than fifty or 100 amino acids. In certain embodiments other modified residues include, but are not limited to phosphorylated, sulfated, sulfonated, or acylated version of Ser, Thr, Gly, or Ala.

The binding affinity of the peptides disclosed herein for calcified surfaces cam be controlled by altering the composition and number of repeats. For example, inclusion of one or more Asp-Ser-Ser repeats will increase the binding affinity of the peptide, because this sequence exhibits the highest affinity of any of the repeats tested. The binding affinity of the peptide may also be increased by increasing the number of three amino acid repeats. Peptides containing more than six repeats generally exhibit greater binding affinity than those with fewer repeats. In certain embodiments, the peptides disclosed herein may have a binding affinity (KA) for hydroxyapatite of greater than 15,000 M⁻¹. In certain embodiments, this binding affinity may be greater than 50,000 M⁻¹, in other embodiments greater than 100,000 M⁻¹, in other embodiments greater than 200,000 M⁻¹, and in other embodiments greater than 300,000 M⁻¹.

In certain embodiments, the peptides can contain one or more additional amino acids that are not part of a three amino acid repeat sequence. For example, in certain embodiments, the repeat portion of the peptide may be fused to an amino acid sequence having an additional functionality e.g. to provide sites (e.g., —SH, OH, COOH, NH₂, etc.) for chemical conjugation to one or more active agents, to function as a linker, to provide biological activity, and the like. In certain of these embodiments, the repeat portion of the peptide can be fused to the additional amino acid sequence via a linker sequence, such as for example a triglycine sequence.

In certain embodiments, the peptides disclosed herein comprise or consist of one or more subunit sequence according to the formula: (X-Y-Z)ₙ, where X is an amino acid selected from aspartic acid (Asp), glutamic acid (Glu), asparagine (Asn), alanine (Ala) and glutamine (Gln), Y and Z are amino acids selected from alanine (Ala), serine (Ser), threonine (Thr), phosphoserine (pSer), phosphothreonine (pThr), and their derivatives, and n is a number between 1 and 100, preferably ranging from about 2 to about 50 or 100, more preferably ranging from about 4 to about 20. In certain embodiments, n ranges from 1 to 15, in other embodiments, n ranges from 1 to 10, and in certain embodiments n ranges from 3 to 4, 6, or 8. In certain embodiments, n is 1, 2, 3, 4, 5, 6, 7, 8, 9, 10, 11, 12, 13, 14, 15, 16, 17, 18, 19, 20, 21, 22, 23, 24, or 25. Illustrative, but non-limiting examples of suitable thre amino acid “DSS” (“XYZ”) subunits are shown in Table 1.

TABLE 1
Illustrative “DSS” (XYZ) subunits.

| X | Y | Z | X | Y | Z |
|---|---|---|---|---|---|
| Asp | Ala | Ala | Asn | Thr | pSer |
| Asp | Ala | Ser | Asn | Thr | pThr |
| Asp | Ala | Thr | Asn | pSer | Ala |
| Asp | Ala | pSer | Asn | pSer | Ser |
| Asp | Ala | pThr | Asn | pSer | Thr |
| Asp | Ser | Ala | Asn | pSer | pSer |
| Asp | Ser | Ser | Asn | pSer | pThr |
| Asp | Ser | Thr | Asn | pThr | Ala |
| Asp | Ser | pSer | Asn | pThr | Ser |
| Asp | Ser | pThr | Asn | pThr | Thr |
| Asp | Thr | Ala | Asn | pThr | pSer |
| Asp | Thr | Ser | Asn | pThr | pThr |
| Asp | Thr | Thr | Ala | Ala | Ala |
| Asp | Thr | pSer | Ala | Ala | Ser |
| Asp | Thr | pThr | Ala | Ala | Thr |
| Asp | pSer | Ala | Ala | Ala | pSer |
| Asp | pSer | Ser | Ala | Ala | pThr |
| Asp | pSer | Thr | Ala | Ser | Ala |
| Asp | pSer | pSer | Ala | Ser | Ser |
| Asp | pSer | pThr | Ala | Ser | Thr |
| Asp | pThr | Ala | Ala | Ser | pSer |
| Asp | pThr | Ser | Ala | Ser | pThr |
| Asp | pThr | Thr | Ala | Thr | Ala |
| Asp | pThr | pSer | Ala | Thr | Ser |
| Asp | pThr | pThr | Ala | Thr | Thr |
| Glu | Ala | Ala | Ala | Thr | pSer |
| Glu | Ala | Ser | Ala | Thr | pThr |
| Glu | Ala | Thr | Ala | pSer | Ala |
| Glu | Ala | pSer | Ala | pSer | Ser |
| Glu | Ala | pThr | Ala | pSer | Thr |
| Glu | Ser | Ala | Ala | pSer | pSer |
| Glu | Ser | Ser | Ala | pSer | pThr |
| Glu | Ser | Thr | Ala | pThr | Ala |
| Glu | Ser | pSer | Ala | pThr | Ser |
| Glu | Ser | pThr | Ala | pThr | Thr |
| Glu | Thr | Ala | Ala | pThr | pSer |
| Glu | Thr | Ser | Ala | pThr | pThr |
| Glu | Thr | Thr | Gln | Ala | Ala |
| Glu | Thr | pSer | Gln | Ala | Ser |
| Glu | Thr | pThr | Gln | Ala | Thr |
| Glu | pSer | Ala | Gln | Ala | pSer |
| Glu | pSer | Ser | Gln | Ala | pThr |
| Glu | pSer | Thr | Gln | Ser | Ala |
| Glu | pSer | pSer | Gln | Ser | Ser |
| Glu | pSer | pThr | Gln | Ser | Thr |
| Glu | pThr | Ala | Gln | Ser | pSer |
| Glu | pThr | Ser | Gln | Ser | pThr |
| Glu | pThr | Thr | Gln | Thr | Ala |
| Glu | pThr | pSer | Gln | Thr | Ser |
| Glu | pThr | pThr | Gln | Thr | Thr |
| Asn | Ala | Ala | Gln | Thr | pSer |
| Asn | Ala | Ser | Gln | Thr | pThr |
| Asn | Ala | Thr | Gln | pSer | Ala |
| Asn | Ala | pSer | Gln | pSer | Ser |
| Asn | Ala | pThr | Gln | pSer | Thr |
| Asn | Ser | Ala | Gln | pSer | pSer |
| Asn | Ser | Ser | Gln | pSer | pThr |
| Asn | Ser | Thr | Gln | pThr | Ala |
| Asn | Ser | pSer | Gln | pThr | Ser |
| Asn | Ser | pThr | Gln | pThr | Thr |
| Asn | Thr | Ala | Gln | pThr | pSer |
| Asn | Thr | Ser | Gln | pThr | pThr |
| Asn | Thr | Thr | | | |

In various embodiments, conservative amino acid substitutions for each position (X, and/or Y, and/or Z) are contemplated.

The subunits can be repeated contiguously (e.g., 8DSS: DSS DSS DSS DSS DSS DSS DSS DSS, SEQ ID NO:6), or, in certain embodiments, one or more subunts can be interspersed with one or more other subunits (e.g., DSS NSS DSS NSS (SEQ ID NO:19), and the like), and/or with non-subunit sequences. In certain embodiments domains comprising one or more subunits (e.g., n=1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, etc.) can be joined to each other to form calcium-binding agents comprising multiple calcium-binding domains. The domains can be joined to each directly, or via a chemical linker to form conjugates or they can be joined via a peptide linker(s) to form a multi-domain peptide/protein.

In certain embodiments, the multiple binding domain moiety comprises at least two domains, at least three domains, at least four domains, at least five domains, at least six domains or more. The calcium binding domains comprising such a construct can be the same or different

As indicated above, various calcium-binding domains comprising such a construct can be joined directly to each other, or two, or more of such domains can be attached to each other via a linker. An illustrative, but non-limiting, list of suitable linkers is provided in Table 3.

In certain embodiments, the calcium binding peptide is unphosphorylated. In certain embodiments the calcium binding peptide is partially or fully phosphorylated. In certain embodiments the peptide is an “L” peptide, while in other embodiments, the peptide is a “D” peptide. In certain embodiments the peptide is a beta peptide.

The foregoing binding moieties are illustrative and non-limiting. Using the teaching provided herein, compositions comprising numerous other binding moieties will be available to one of skill in the art.

Active Agents

In various embodiments the binding moiety is attached to one or more active agent(s) that provide a desirable action when administered to a tooth. Illustrative, but non-limiting active agents include, but are not limited to compositions to whiten and/or brighten a tooth, compositions to protect a tooth surface, compositions to restore and/or remineralize a tooth, compositions to reduce plaque or to inhibit plaque formation, compositions to desensitize a tooth, compositions to remove staining or to prevent staining of a tooth, and the like.

White Colorants.

Suitable colorants that may be used as active agents in the dental care compositions contemplated herein for the whitening and/or brightening of teeth include, but are not limited to, white pigments such as titanium dioxide, titanium dioxide nanoparticles, white minerals such as hydroxyapatite, and zircon (zirconium silicate), and the like. An illustrative, but non-limiting list of suitable white colorants is provided in Table 2.

TABLE 2
Illustrative, but non-limiting, examples of white colorants.

| | Composition/ | Microscopic | Other | |
|---|---|---|---|---|
| Pigment | CI Number | characteristics | Characteristics | Health |
| Aluminum | Al(OH)₃ | Fine grains, colorless in | Fluoresces purple | No significant |
| trihydrate | Pigment White | plane-polarized light; no | | hazards |
| | 24; CI7702 | birefringence | | |
| Barite | Barium sulfate, | Difficult to see in | Often used as an | No significant |
| | BaSO₄ | meltmount; low | extender in | hazards |
| | Natural pigment | birefringence under | conjunction with | |
| | white 22; | cross polars, rotating the | other white | |
| | synthetic white | stage may cause the | pigments | |
| | 21, CI77120 | particles to twinkle | | |
| Chalk | Calcium | Small irregular shaped | May fluoresce a | No significant |
| (whiting) | carbonate, CaCO₃ | particles (0.1-10 μm); | medium purple | hazards |
| | Pigment White | high birefringence with | color; reacts with | |
| | 18; CI 77220 | strong interference | acids to evolve | |
| | | colors | carbon dioxide | |
| Gypsum | Calcium sulfate | low birefringence; | Fluoresces purple | No significant |
| | dyhydrate, CaSO₄2H₂O | euhedral shaped crystals | | hazards |
| | Pigment White 25 | with inclusions | | |
| Kaolin | Al₂Si₂O₅(OH₄) | Translucent and | Fluoresces pale | No significant |
| | Pigment white 19 | colorless with moderate | white | hazards |
| | CI 77005 | relief; under crossed | | |
| | | polars particles have | | |
| | | low birefringence. | | |
| Lithopone | ZnS (30%): | Very fine particles (0.3-0.5 μm) | Dissolves in | No significant |
| | BaSO₄ (70%) | | HCL releasing | hazards |
| | Pigment White 5 | | sulfur fumes; can | |
| | | | darken in the | |
| | | | presence of iron | |
| Magnesite | magnesium | Translucent, colorless, | Soluble in acids | Nontoxic, |
| | carbonate, | angular crystals; high | | ingestion has a |
| | MgCO₃ | birefringence under | | laxative effect |
| | Pigment white 18 | crossed polars; | | |
| | | extinction is complete | | |
| | | and straight. | | |
| Silica/quartz | Silicon dioxide, | Chonchoidal fracture; | Dissolves in | No significant |
| | SiO₂ | slightly birefringent. | hydrofluoric acid | hazards |
| | Pigment white 27 | Ground glass is | | |
| | | isotropic. | | |
| Talc | Hydrated | Ground particles can be | Insoluble in | No significant |
| | magnesium | very small (2 μm); high | water, acids or | hazards |
| | silicate, | birefringence | alkalis | |
| | Mg₃Si₄O₁₀(OH)₂ | | | |
| | Pigment white 26 | | | |
| Titanium | TiO₂ | Small round particles | Weak white | No significant |
| dioxide | | (0.2-0.3 μm) high | fluorescence | hazards |
| (anatase) | | birefringence under | | |
| | | crossed polars | | |
| Titanium | TiO₂ | Small round or prism | Fluoresces gray | No significant |
| dioxide | Pigment white 6, | particles (0.2-0.5 μm); | or dark purple | hazards |
| (rutile) | CI77891 | high birefringence and | | |
| | | interference colors | | |
| Zinc white | Zinc oxide, ZnO₄ | Very fine crystalline | Fluoresces | Inhalation or |
| | Pigment white 4, | grains with low | yellow in | ingestion of |
| | CI77947 | birefringence and first | longwave UV | dust may |
| | | order interference colors | | cause irritation |

In certain embodiments the whitening and/or brightening agent(s) can include fluorescent molecules, pigments, or dyes, such as FITC, Rhodamine, Blue No. 1, Yellow No. 5, blue fluorescent molecules, luminol, isoluminol, other luminescent molecules, and the like.

In various embodiments whitening agents can include a bleach. Peroxide is a particularly suitable bleaching agent. The efficacy of peroxide compounds in oral hygiene has been long recognized. In certain embodiments the peroxide is carbamide peroxide (CO(NH₂)₂H₂O₂), also called urea hydrogen peroxide, hydrogen peroxide carbamide, and perhydrol-urea. Also, peroxide salts of the alkali or alkaline earth metals are contemplated.

Enzymes.

In various embodiments the active agent comprising the dental care composition(s) contemplated herein comprises one or more enzymes having a desirable activity when applied to tooth surface. Suitable enzymes may be naturally occurring or recombinant enzymes including, but not limited to, oxidases, peroxidases, proteases, lipases, glycosidases, esterases, and polysaccharide hydrolases.

Anti-Plaque Agents

In various embodiments the active agent comprising the dental care composition(s) contemplated herein comprises one or more anti-plaque agents. Illustrative anti-plaque agents include, but are not limited to, fluoride ion sources and anti-microbial agents. Suitable anti-microbial agents include, but are not limited to magainins, and cecropins; microbiocides such as triclosan, chlorhexidine, quaternary ammonium compounds, chloroxyylenol, chloroxyethanol, phthalic acid and its salts, and thymol.

Desensitizing Agents

In various embodiments the active agent comprising the dental care composition(s) contemplated herein comprises one or more desensitizing agents. Numerous desensitizing agents are known to those of skill in the art. Illustrative desensitizers include, but are not limited to dipotassium oxalate (found at 3% in REME•SENSET™ desensitizer), 2-hydroxyethyl methacrylate, potassium nitrate (found at 5% in toothpastes like SENSODYNE®), and the like.

Remineralization Agents

In various embodiments the active agent comprising the dental care composition(s) contemplated herein comprises one or more remineralization agents. Numerous remineralization agents are known to those of skill in the art. Illustrative remineralization agents suitable for use as active agents in the dental care compositions contemplated herein include, but are not limited to fluoride and fluoride containing materials, casein phosphopeptide-Amorphous Calcium Phosphate (CPP-AP marketed as RECALDENT®), ethylenediamine tetramethylenephosphonic acid or water soluble salt thereof (see, e.g., U.S. Pat. No. 4,177,258), dicalcium phosphate (see, e.g., European Patent EP 0040938), combinations of one or more of coenzyme Q10, selenium and bromine (see, e.g., U.S. Pat. No. 6,372,198, calcium forms of zeolite (see, e.g., U.S. Patent Publication No: US 2008/152598), various organophosphate compounds (see, e.g., Mexico Patent No: MX 2010014220), calcium peroxymonophosphate and/or calcium diperoxymonophosphate (see, e.g., U.S. Patent Publication No: US 2011/142768), water soluble phosphate, particularly a pyrophosphate or tripolyphosphate (see, e.g., PCT Publication No: WO 9319728), calcium lactate (see, e.g., PCT Publication No: WO 0042861), starch phosphate, maltodextrin phosphate, reducing maltodextrin phosphate, oligosaccharide phosphate (see, e.g., PCT Publication No: WO 2005003753), calcium glycerophosphate and nanocrystalline calcium hydroxyapatite (see, e.g., EA 200801499), a combination of calcium glycerophosphate 0.1-3.0, a source of magnesium ions (e.g., magnesium chloride, magnesium sulfate or magnesium) 0.01-0.50 (see, e.g., EA 200800988), and the like. Additional suitable remineralization agent(s) are known to those of skill in the art and include, but are not limited to those described in U.S. Pat. Nos. 6,669,931, 6,485,708, 6,451,290, 6,372,198, 6,214,321, 6,159,448, 6,120,754, 6,036,944, 5,866,102, 5,833,957, 5,817,296, 5,645,853, 5,614,175, 5,603,922, 5,571,502, 4,606,912, and 4,397,837.

The foregoing active agents are illustrative and not limiting. Using the teaching provided herein dental care compositions comprising other active agents will be readily available to one of skill in the art.

### Preparation of a Dental Care Composition.

Binding Moieties.

The non-peptide binding moieties described herein are commercially available and/or can be prepared by synthetic methods well known to those of skill in the art.

The binding moieties that are or comprise peptides or proteins described herein can be chemically synthesized using standard chemical peptide synthesis techniques or, particularly where the peptide does not comprise “D” amino acid residues, the peptide can be recombinantly expressed. Where the “D” polypeptides are recombinantly expressed, a host organism (e.g. bacteria, plant, fungal cells, etc.) can be cultured in an environment where one or more of the amino acids is provided to the organism exclusively in a D form. Recombinantly expressed peptides in such a system then incorporate those D amino acids.

In certain embodiments, D amino acids can be incorporated in recombinantly expressed peptides using modified amino acyl-tRNA synthetases that recognize D-amino acids.

In certain embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In one embodiment, the peptides can be synthesized by the solid phase peptide synthesis procedure using a benzhyderylamine resin (Beckman Bioproducts, 0.59 mmol of NH₂/g of resin) as the solid support. The COOH terminal amino acid (e.g., t-butylcarbonyl-Phe) is attached to the solid support through a 4-(oxymethyl)phenacetyl group. This is a more stable linkage than the conventional benzyl ester linkage, yet the finished peptide can still be cleaved by hydrogenation. Transfer hydrogenation using formic acid as the hydrogen donor can be used for this purpose.

It is noted that in the chemical synthesis of peptides, particularly peptides comprising D amino acids, the synthesis usually produces a number of truncated peptides in addition to the desired full-length product. Thus, the peptides are typically purified using, e.g., HPLC.

D-amino acids, beta amino acids, non-natural amino acids, and the like can be incorporated at one or more positions in the peptide simply by using the appropriately derivatized amino acid residue in the chemical synthesis. Modified residues for solid phase peptide synthesis are commercially available from a number of suppliers (see, e.g., Advanced Chem Tech, Louisville; Nova Biochem, San Diego; Sigma, St Louis; Bachem Calif. Inc., Torrance, etc.). The D-form and/or otherwise modified amino acids can be completely omitted or incorporated at any position in the peptide as desired. Thus, for example, in certain embodiments, the peptide can comprise a single modified acid, while in other embodiments, the peptide comprises at least two, generally at least three, more generally at least four, most generally at least five, preferably at least six, more preferably at least seven or even all modified amino acids. In certain embodiments, essentially every amino acid is a D-form amino acid.

As indicated above, the peptides and/or dental care compositions comprising peptides can also be recombinantly expressed. Accordingly, in certain embodiments, the binding moieties, and/or fusion proteins are synthesized using recombinant expression systems. Generally this involves creating a DNA sequence that encodes the desired peptide or fusion protein, placing the DNA in an expression cassette under the control of a particular promoter, expressing the peptide or fusion protein in a host, isolating the expressed peptide or fusion protein and, if required, renaturing the peptide or fusion protein.

DNA encoding the peptide(s) or fusion protein(s) described herein can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding the peptides or fusion proteins described herein can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will typically be operably linked to appropriate expression control sequences for each host. For E. coli this can include a promoter such as the T7, tip, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant peptide(s) or fusion protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred.

One of skill in the art would recognize that after chemical synthesis, biological expression, or purification, the peptide(s) or fusion protein(s) may possess a conformation substantially different than desired native conformation. In this case, it may be necessary to denature and reduce the peptide or fusion protein and then to cause the molecule to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the peptide(s) and/or fusion protein(s) proteins without diminishing their biological activity. Some modifications may be made to facilitate the cloning, expression, or incorporation of the binding moiety and/or the active agent into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Active Agents

Where the active agent is or comprises a peptide (or protein) the peptide or protein can be chemically synthesized or recombinantly expressed, e.g., as described above.

Non-peptide active agent(s) described herein are commercially available and/or can be prepared by synthetic methods well known to those of skill in the art (e.g., as taught in patents, patent publications, and the scientific literature).

Joining Binding Moieties to Active Agents.

Chemical Conjugation.

The dental care compositions can be made by joining one or more of the binding moieties described herein to one or active agents. In certain embodiments the binding moieties are attached directly to the active agent(s) via naturally occurring reactive groups or the binding moiety and/or the active agent(s) can be functionalized to provide such reactive groups.

The coupling interaction may be a covalent bond or a non-covalent interaction, such as hydrogen bonding, electrostatic interaction, hydrophobic interaction, or Van der Waals interaction. In the case of a non-covalent interaction, the dental care composition may be prepared by mixing the binding moiety with the active agent and the optional spacer (if used) and allowing sufficient time for the interaction to occur. The unbound materials may be separated from the resulting dental care composition adduct using methods known in the art, for example, gel permeation chromatography.

In certain embodiments the binding moieties are attached directly to the active agent(s) (or to linkers) via naturally occurring reactive groups or the binding moiety and/or the active agent(s) can be functionalized to provide such reactive groups.

In various embodiments the binding moieties are attached to active agent(s) via one or more linking agents. Thus, in various embodiments the binding moieties and the active agent(s) can be conjugated via a single linking agent or multiple linking agents. For example, the binding moiety and the active agent can be conjugated via a single multifunctional (e.g., bi-, tri-, or tetra-) linking agent or a pair of complementary linking agents. In another embodiment, the binding moiety and the active agent are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets. In some embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions useful in forming chimeric moieties include those that are well known in the art of bioconjugate chemistry. Currently favored classes of reactions available include those that proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C.

In certain embodiments, the linking agent comprises a chelator. For example, the chelator comprising the molecule, DOTA (DOTA=1,4,7,10-tetrakis(carboxymethyl)-1,4,7,10-tetraazacyclododecane), can readily be coupled to a number of molecules. Other suitable chelates are known to those of skill in the art, for example, 1,4,7-triazacyclononane-N,N′,N″-triacetic acid (NOTA) derivatives being among the most well-known (see, e.g., Lee et al. (1997) Nucl Med. Biol. 24: 2225-23019).

A “linker” or “linking agent” as used herein, is a molecule that is used to join two or more molecules. In certain embodiments the linker is typically capable of forming covalent bonds to both molecule(s) (e.g., the binding moiety and the active agent). Suitable linkers are well known to those of skill in the art and include, but are not limited to, straight or branched-chain carbon linkers, heterocyclic carbon linkers, or peptide linkers. In certain embodiments the linkers can be joined to the constituent amino acids through their side groups (e.g., through a disulfide linkage to cysteine). However, in certain embodiments, the linkers will be joined to the alpha carbon amino and carboxyl groups of the terminal amino acids.

A bifunctional linker having one functional group reactive with a group on one molecule (e.g., a binding peptide), and another group reactive on the other molecule (e.g., an antimicrobial peptide), can be used to form the desired conjugate. Alternatively, derivatization can be performed to provide functional groups. Thus, for example, procedures for the generation of free sulfhydryl groups on peptides are also known (See U.S. Pat. No. 4,659,839).

In certain embodiments the linking agent is a heterobifunctional crosslinker comprising two or more different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In one embodiment, the heterobifunctional crosslinker is SMCC.

Suitable linkers/coupling agents also include, but are not limited to, carbodiimide coupling agents, diacid chlorides, diisocyanates and other difunctional linker/coupling reagents that are reactive available amine and/or carboxylic acid groups on the binding moiety (e.g., terminal amine or carboxylic acid groups on peptide binding moieties) and to amine, carboxylic acid, or alcohol groups on the active agent. Common coupling agents include, but are not limited to carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups for coupling to alcohol, and amine groups.

Additionally, it may be necessary to protect reactive amine or carboxylic acid groups on the binding moiety and/or the active agent to produce the desired structure for the dental care compositon. The use of protecting groups for amino acids, such as t-butyloxycarbonyl (t-Boc), are well known in the art. In some cases it may be necessary to introduce reactive groups, such as carboxylic acid, alcohol, amine, or aldehyde groups, on the on the binding moiety and/or the active agent. These modifications may be done using routine chemistry such as oxidation, reduction and the like, which is well known in the art.

In certain embodiments it may be desirable to couple the binding moiety to the active agent via a linker. The linker can serves to separate binding moiety from the active agent to ensure that the agent does not interfere with the binding of the binding moiety to a tooth surface. The linker may be any of a variety of molecules including, but not limited to alkyl chains, phenyl compounds, ethylene glycol, amides, esters, peptide linkers, and the like.

Illustrative linkers can be hydrophilic or hydrophobic and have a chain length from 1 to about 100 atoms, more preferably, from 2 to about 30 atoms. Examples of illustrative linkers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains.

The linker may be covalently attached to the binding moiety and to the active agent(s) using any of the coupling chemistries described above. In certain embodiments in order to facilitate incorporation of the linker, a bifunctional cross-linking agent that contains a linker and reactive groups at both ends for coupling to the binding moiety and to the active agent may be used. Suitable bifunctional cross-linking agents are well known in the art and include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents include, but are not limited to compounds having the following structure:

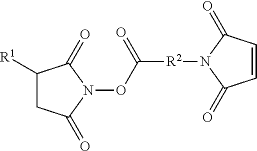

R¹ is H or a substituent group such as —SO₃Na, —NO₂, or —Br; and R² is a linker (e.g., an alkyl, an alkaryl, etc.) such as —CH₂CH₂ (ethyl), —(CH₂)₃ (propyl), —(CH₂)₃C₆H₅ (propyl phenyl), and the like. One illustrative, but non-limiting example of such a heterobifunctional cross-linking agent is 3-maleimidopropionic acid N-hydroxysuccinimide ester. The N-hydroxysuccinimide ester group of these reagents reacts with amine or alcohol groups, while the maleimide group reacts with thiol. A thiol group may be incorporated into a peptide by adding a cysteine group to at least one end of the peptide sequence (i.e., the C-terminus or N-terminus). Several linker amino acid residues, such as glycine, may be incorporated between the binding peptide sequence and the terminal cysteine to separate the reacting thiol group from the binding sequence.

Additionally, the linker can comprise a peptide composed of any amino acid and mixtures thereof. Illustrative peptide linkers are composed of the amino acids glycine, alanine, and serine, and mixtures thereof (see, e.g., Table 3). In various embodiments the peptide linker may be from 1 to about 50 amino acids, or from 1 to about 40 amino acids, or from 1 to about 30 amino acids, or from 1 to about 20 amino acids, or from 1, 2, or 3 up to about 15 amino acids. These peptide linkers may be linked to binding moieity and/or to the active agent by any method known in the art. For example, where both the binding moiety and the active agent comprise peptide(s), the entire dental care composition may be prepared using the standard peptide synthesis methods or expressed recombinantly, e.t., as described supra.

In certain embodiments the binding moiety and peptide linker and/or active agent can may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid terminal groups on the peptides. In certain embodiments the linker may also be a combination of a peptide linker and an organic linker molecule, which may be prepared using the methods described above.

It may also be desirable to have multiple binding moieties coupled to the active agent to enhance the interaction between the active agent(s) and the tooth. Either multiple copies of the binding moiety or a combination of different binding moieties may be used. In the case of large active agents, (e.g., particle emulsions), a large number of binding moieties, e.g., up to about 1,000, may be coupled to the active agent. A smaller number of binding moieties can be coupled to smaller active agents.

Fusion Proteins.

In certain embodiments where the binding moiety and active agent are both peptides or both comprise peptides, the chimeric moiety can be chemically synthesized or recombinantly expressed as a fusion protein (i.e, a chimeric fusion protein).

In certain embodiments the chimeric fusion proteins are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

In certain embodiments, DNA encoding fusion proteins of the present invention may be cloned using DNA amplification methods such as polymerase chain reaction (PCR). Thus, for example, the nucleic acid encoding a binding antibody, a binding peptide, and the like is PCR amplified, using a sense primer containing the restriction site for NdeI and an antisense primer containing the restriction site for HindIII. This produces a nucleic acid encoding the binding sequence and having terminal restriction sites. Similarly an active agent and/or active agent/linker/spacer can be provided having complementary restriction sites. Ligation of sequences and insertion into a vector produces a vector encoding the fusion protein.

While, in certain embodiments, the binding moieties and active agent(s) can be directly joined together, one of skill will appreciate that they can be separated by a peptide spacer/linker consisting of one or more amino acids. Generally the spacer will have no specific biological activity other than to join the proteins or to preserve some minimum distance or other spatial relationship between them. However, the constituent amino acids of the spacer may be selected to influence some property of the molecule such as the folding, net charge, or hydrophobicity.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the fusion protein may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the binding molecule into a fusion protein. Such modifications are well known to those of skill in the art and include, for example, a methionine added at the amino terminus to provide an initiation site, or additional amino acids placed on either terminus to create conveniently located restriction sites or termination codons.

As indicated above, in various embodiments a peptide linker/spacer is used to join the one or more binding moieties to one or more active agent(s). In various embodiments the peptide linker is relatively short, typically less than about 10 amino acids, preferably less than about 8 amino acids and more preferably about 3 to about 5 amino acids. Suitable illustrative linkers include, but are not limited to PSGSP ((SEQ ID NO:20), ASASA (SEQ ID NO: 21), or GGG (SEQ ID NO: 22). In certain embodiments longer linkers such as (GGGGS)₃ (SEQ ID NO:23) can be used. Illustrative peptide linkers and other linkers are shown in Table 3.

TABLE 3
Illustrative peptide and non-peptide linkers.

| | SEQ |
|---|---|
| Linker | ID NO: |
| AAA | |
| GGG | |
| SAT | |
| PYP | |
| SGG | |
| ASA | |
| GGGG | 24 |
| GGSGGS | 25 |
| PSPSP | 26 |
| ASASA | 27 |
| PSPSP | 28 |
| KKKK | 29 |
| RRRR | 30 |
| GGGGS | 31 |
| GGGGS GGGGS | 32 |
| GGGGS GGGGS GGGGS | 33 |
| GGGGS GGGGS GGGGS GGGGS | 34 |
| GGGGS GGGGS GGGGS GGGGS GGGGS | 35 |
| GGGGS GGGGS GGGGS GGGGS GGGGS GGGGS | 36 |
| 2-nitrobenzene or O-nitrobenzyl | |
| Nitropyridyl disulfide | |
| Dioleoylphosphatidylethanolamine (DOPE) | |
| S-acetylmercaptosuccinic acid | |
| 1, 4, 7, 10-tetraazacyclododecane-1, 4, 7, | |
| 10-tetracetic acid (DOTA) | |
| β-glucuronide and β-glucuronide variants | |
| Poly(alkylacrylic acid) | |
| Benzene-based linkers (for example: 2,5- | |
| Bis(hexyloxy)-1,4-bis[2,5-bis(hexyloxy)-4- | |
| formyl-phenylenevinylene] benzene) and | |
| like molecules | |
| Disulfide linkages | |
| Poly(amidoamine) or like dendrimers linking | |
| multiple target and killing peptides in one | |
| molecule | |
| Carbon nanotubes | |
| Hydrazone and hydrazone variant linkers | |
| PEG of any chain length | |
| Succinate, formate, acetate butyrate, | |
| other like organic acids | |
| Aldols, alcohols, or enols | |
| Peroxides | |
| alkane or alkene groups of any chain length | |
| One or more porphyrin or dye molecules | |
| containing free amide and carboxylic | |
| acid groups | |
| One or more DNA or RNA nucleotides, | |
| including polyamine and | |
| polycarboxyl-containing variants | |
| Inulin, sucrose, glucose, or other single, | |
| di or polysaccharides | |
| Linoleic acid or other polyunsaturated | |
| fatty acids | |
| Variants of any of the above linkers | |
| containing halogen or thiol groups | |

(Allamino-acid-based linkers could be L, D, combinatiobns of L and D forms, β-form, and the like)

### Dental Care Formulations

In various embodiments the dental care compositions (e.g., those compositions described above and in the claims) are formulated into a dental formulations (e.g., dental care products). Illustrative dental care products include, but are not limited to, toothpaste, dental cream, gel or tooth powder, mouth wash, mouth lozenges, mouth sprays or aerosols, tooth whitening strips, toothbrush bristles, toothpicks, and dental floss. The dental care formulations typically comprise an effective amount of the dental care composition(s) in an orally acceptable carrier medium. An effective amount of the dental care composition for use in a dental care formulation (e.g., oral care product) may vary depending on the type of product. Typically, the effective amount of the dental care composition is a proportion that ranges from about 0.001% to about 90%, or from about 0.01% to about 85%, or from about 0.1% to about 80%, or from about 0.01%, or from about 0.1%, or from about 1%, or from about 2% up to about 70% or up to about 60%, or up to about 50%, or up to about 40%, or up to about 30%, or up to about 20%, or up to about 15%, or up to about 10% by weight of the total product composition.

The dental care formulation may contain one type of dental care composition, or a mixture of different dental care compositions.

Components of orally acceptable carrier media are well known to those of skill in the art and are described for example, in U.S. Pat. Nos. 6,740,311, 6,706,256, and 6,264,925. For example, in addition to the dental care composition(s) described herein, the dental care formulation may contain one or more of the following: abrasives, surfactants, chelating agents, fluoride sources, thickening agents, buffering agents, solvents, humectants, carriers, bulking agents, and the like. In certain embodiments, the formulation may optionally comprise one or more flavorants (e.g., oil of wintergreen, oil of peppermint, oil of spearmint, menthol, methyl salicylate, eucalyptol, vanillin, cinnamon, and the like).

The formulations may be prepared using standard techniques that are well known in the art. The formulation of such health products is well known to those of skill, and the dental care composition(s) are typically simply added to such formulations in an effective dose.

For example, toothpaste formulations are well known to those of skill in the art. Typically such formulations are mixtures of abrasives and surfactants, anticaries agents, such as fluoride; tartar control ingredients, such as tetrasodium pyrophosphate and methyl vinyl ether/maleic anhydride copolymer, pH buffers; humectants, to prevent dry-out and increase the pleasant mouth feel; and binders, to provide consistency and shape (see, e.g., Table 4). Binders keep the solid phase properly suspended in the liquid phase to prevent separation of the liquid phase out of the toothpaste. They also provide body to the dentifrice, especially after extrusion from the tube onto the toothbrush.

TABLE 4
Typical components of toothpaste.

| Ingredients | Wt % |
|---|---|
| Humectants | 40-70 |
| Water | 0-50 |
| Buffers/salts/tartar control | 0.5-10 |
| Organic thickeners (gums) | 0.4-2 |
| Inorganic thickeners | 0-12 |
| Abrasives | 10-50 |
| Actives (e.g., triclosan) | 0.2-1.5 |
| Surfactants | 0.5-2 |
| Flavor and sweetener | 0.8-1.5 |

Fluoride sources provide 1000-15000 ppm fluorine.

Table 5 lists typical ingredients used in formulations; the final combination will depend on factors such as ingredient compatibility and cost, local customs, and desired benefits and quality to be delivered in the product. It will be recognized that one or more antimicrobial peptides and/or chimeric constructs described herein can simply be added to such formulations or used in place of one or more of the other ingredients.

TABLE 5
List of typical ingredients.

| | Inorganic | | | | Tartar Control |
|---|---|---|---|---|---|
| Gums | Thickeners | Abrasives | Surfactants | Humectants | Ingredient |
| Sodium | Silica | Hydrated | Sodium | Glycerine | Tetrasodium |
| carboxymethyl | thickeners | silica | lauryl | | pyrophosphate |
| cellulose | | | sulfate | | |
| Cellulose ethers | Sodium | Dicalcium | Sodium | Sorbitol | Gantrez S-70 |
| | aluminum | phosphate | N-lauryl | | |
| | silicates | digydrate | sarcosinate | | |
| Xanthan Gum | Clays | Calcium | Pluronics | Propylene | Sodium tri- |
| | | carbonate | | glycol | polyphosphate |
| Carrageenans | | Sodium | | Xylitol | |
| | | bicarbonate | | | |
| Sodium alginate | | Calcium | Sodium | Polyethylene | |
| | | pyrophosphate | lauryl | glycol | |
| | | | sulfoacetate | | |
| Carbopols | | Alumina | | | |

One illustrative formulation described in U.S. Pat. No. 6,113,887 comprises (1) a water-soluble bactericide selected from the group consisting of pyridinium compounds, quaternary ammonium compounds and biguanide compounds in an amount of 0.001% to 5.0% by weight, based on the total weight of the composition; (2) a cationically-modified hydroxyethylcellulose having an average molecular weight of 1,000,000 or higher in the hydroxyethylcellulose portion thereof and having a cationization degree of 0.05 to 0.5 mol/glucose in an amount of 0.5% to 5.0% by weight, based on the total weight of the composition; (3) a surfactant selected from the group consisting of polyoxyethylene polyoxypropylene block copolymers and alkylolamide compounds in an amount of 0.5% to 13% by weight, based on the total weight of the composition; and (4) a polishing agent of the non-silica type in an amount of 5% to 50% by weight, based on the total weight of the composition. In certain embodiments, the dental care compositions herein can be simply added to such a formulation.

Similarly, mouthwash formulations are also well known to those of skill in the art. Thus, for example, mouthwashes containing sodium fluoride are disclosed in U.S. Pat. Nos. 2,913,373, 3,975,514, and 4,548,809, and in US Patent Publications US 2003/0124068 A1, US 2007/0154410 A1, and the like. Mouthwashes containing various alkali metal compounds are also known: sodium benzoate (WO 9409752); alkali metal hypohalite (US 20020114851A1); chlorine dioxide (CN 1222345); alkali metal phosphate (US 2001/0002252 A1, US 2003/0007937 A1); hydrogen sulfate/carbonate (JP 8113519); cetylpyridium chloride(CPC) (see, e.g., U.S. Pat. No. 6,117,417, U.S. Pat. No. 5,948,390, and JP 2004051511). Mouthwashes containing higher alcohol (see, e.g., US 2002/0064505 A1, US 2003/0175216 A1); hydrogen peroxide (see, e.g., CN 1385145); CO₂ gas bubbles (see, e.g., JP 1275521 and JP 2157215) are also known. In certain embodiments, these and other mouthwash formulations can further comprise one or more of the dental care compositions described herein.

The foregoing formulations are meant to be illustrative and not limiting. Using teaching provided herein, the dental care compositions described herein can readily be incorporated into other products.

### Methods of Use.

In various embodiments methods of use of the dental care compositions and/or formulations described herein are contemplated. One illustrative use is a method of improving the health and/or appearance of a tooth, by applying the dental care composition(s) and/or formulation(s) described herein the said method comprising contacting said tooth with an effective amount of a dental composition and/or claimed herein and/or a dental care formulation (e.g., oral care product) as described and/or claimed herein. In certain embodiments improving the health and/or appearance of a tooth includes brightening and/or whitening said tooth and the active agent comprising the composition and/or formulation includes a whitening and/or brightening agent. In certain embodiments improving the health and/or appearance of a tooth includes remineralizing and/or preventing demineralization of a tooth and the active agent comprising the composition and/or formulation includes a remineralization agent. In certain embodiments improving the health and/or appearance of a tooth involves desensitizing a tooth and the active agent comprising the composition and/or formulation includes a desensitizing agent. In certain embodiments improving the health and/or appearance of a tooth includes reducing plaque formation and/or removing plaque and the active agent comprising the composition and/or formulation includes an anti-plaque agent. In various embodiments the tooth is a tooth in a human and the method is performed by that person. In certain embodiments the method is performed by a dentist, or dental technician, to a dental patient.

### Kits.

In another embodiment kits are provided for the use of the dental care compositions and/or dental care formulations (e.g., oral care products) described herein. The kits typically comprise a container containing one or more of the dental care compositions and/or dental care formulations herein. In certain embodiments the dental care composition can be provided in a unit dosage formulation.

In certain embodiments the kits comprise one or more dental care formulations as described herein. Illustrative kits, include, but are not limited to one or more of the following: toothpaste, dental cream, gel or tooth powder, mouth wash, mouth lozenges, mouth sprays or aerosols, tooth whitening strips, toothpicks, and/or dental floss comprising one or more of the dental care compositions described herein.

In addition, the kits optionally include labeling and/or instructional materials providing directions for the use of the components thereof. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1: A composition for the whitening, and/or brightening, and/or restoration of a tooth, said composition comprising:
a binding moiety that binds to calcium and/or to tooth enamel and/or to pellicle attached to an active agent selected from the group consisting of a white colorant, a bleach, an enzyme, an anti-plaque agent, a desensitization agent, a remineralization agent, or any combination thereof.

2: The composition of claim 1, wherein a plurality of binding moieties are attached to each active agent.

3: The composition of claim 1, wherein a plurality of active agents are attached to each binding moiety.

4: The composition of claim 1, wherein a single binding moiety is attached to a single active agent.

5: The composition of claim 1, wherein said binding moiety is selected from the group consisting of an enamel matrix proteins (EMP) or enamel-binding fragment thereof, an enamel or calcium binding peptide, and an enamel or calcium binding tannin.

6-7. (canceled)

8: The composition of claim 5, wherein said binding moiety comprises an enamel and/or calcium binding peptide comprising one or more peptide domains said domain(s) comprising the sequence (X-Y-Z)ₙ, wherein:
X is an amino acid selected from the group consisting of aspartic acid, glutamic acid, asparagine, alanine and glutamine or a conservative substitution thereof;
Y and Z are amino acids independently selected from alanine, serine, threonine, phosphoserine, and phosphothreonine, or conservative substitutions thereof; and n is a number from 1 to 100; and
wherein when more than one of said domains is present the domains can be the same or different; and wherein said calcium-binding peptide moiety binds calcium phosphate.

9-12. (canceled)

13: The composition of claim 8, wherein Y and Z are the same amino acid.

14: The composition of claim 13, wherein Y and Z are both serine or a conservative substitution thereof or both phosphoserine or a conservative substitution thereof, or both threonine or a conservative substitution thereof, or both phosphothreonine or a conservative substitution thereof.

15-23. (canceled)

24: The composition of claim 8, wherein X is aspartic acid or a conservative substitution thereof, or glutamic acid or a conservative substitution thereof.

25-33. (canceled)

34: The composition of claim 8, wherein said peptide ranges in length from about 6 to about 60 amino acids.

35. (canceled)

36: The composition of claim 8, wherein n ranges from about 3 up to about 12.

37. (canceled)

38: The composition of claim 8, wherein the amino acid sequence of said peptide comprises a sequence selected from the group consisting of (DSS)₃, (DSS)₄, (DSS)₅, (DSS)₆, (DSS)₇, (DSS)₈, (DSS)₉, and (DSS)₁₀.

39-41. (canceled)

42: The composition of claim 8, wherein said peptide is not phosphorylated.

43-47. (canceled)

48: The composition of claim 1, wherein said active agent comprises a whitening agent and/or a colorant.

49: The composition of claim 48, wherein said whitening agent and/or colorant comprises a bleach or a peroxide.

50. (canceled)

51: The composition of claim 48, wherein said whitening agent and/or colorant is selected from the group consisting of hydroxyapatite, zirconium silicate, titanium dioxide, titanium dioxide nanoparticles silica dioxide, and a white pigment or dye.

52. (canceled)

53: The composition claim 1, wherein said active agent comprises an enzyme selected from the group consisting of an oxidase, a peroxidase, a protease, a lipase, glucosidase, an esterase, and a polysaccharide hydrolase.

54. (canceled)

55: The composition of claim 1, wherein said active agent comprises an agent selected from the group consisting of an anti-plaque agent, a desensitization agent, and a remineralization agent.

56-65. (canceled)

66: The composition of claim 1, wherein said active agent is attached to said binding moiety through a peptide linker.

67-72. (canceled)

73: An oral care formulation comprising an effective amount of the composition of claim 1, wherein said formulation is selected from the group consisting of toothpaste, dental cream, gel, tooth powder, mouth wash, breath freshener, and dental floss.

74-77. (canceled)

78: A method of improving the health and/or appearance of a tooth, said method comprising contacting said tooth with an effective amount of a composition of claim 1.

79-85. (canceled)
